(19) United States
(12) Patent Application Publication
(10) Pub. No.: US 2010/0122363 A1
(43) Pub. Date: May 13, 2010

(54) Certain human genomic DNA associated with total red-green colorblindness

(76) Inventor: Charles Timberlake Zeleny, Baltimore, MD (US)

Correspondence Address: DUANE MORRIS LLP - DC, 505 9th Street, Suite 1000, WASHINGTON, DC, 20004-2166, US

(21) Appl. No.: 12/588,704

(22) Filed: Oct. 26, 2009

Related U.S. Application Data
(63) Continuation of application No. 11/703,096, filed on Feb. 7, 2007, now abandoned.

(51) Int. Cl. C07H 21/04; C12N 5/10; A01K 67/027

(52) U.S. Cl. 800/14; 536/23.5; 435/325

(57) ABSTRACT

Disclosed is human genomic DNA having the following STR marker profile:

| Amel | D3S1358 | VWA | FGA | D8S1179 | D21S11 | D18S51 |
|---|---|---|---|---|---|---|
| M | 15-17 | 16-17 | 21-25 | 12-14 | 29-32.2 | 17-18 |

| D5S818 | D13S317 | D7S820 | CSF1PO | TPOX | TH01 | D16S539 |
|---|---|---|---|---|---|---|
| 11-12 | 11-12 | 8-8 | 10-11 | 8-8 | 7-9.3 | 10-13 |

And the use thereof as a reference for studying variations in the human genome that may be associated with certain genetic traits or diseases.

### FIELD OF THE INVENTION

The present invention is in the field of recombinant DNA technology. More specifically, the invention is directed to a particular, isolated human genomic DNA which is associated with certain genetic characteristics, such as total red-green colorblindness, and methods suitable for identifying polymorphisms in the genome of a human using such genomic DNA, and using such sites to analyze identity, ancestry or genetic traits.

### BACKGROUND OF THE INVENTION

The capacity to genotype a human is of fundamental importance to forensic science, medicine and epidemiology and public health, and to the breeding and exhibition of animals. Such a capacity is needed, for example, to determine the identity of the causative agent of an infectious disease or to determine whether two individuals are related.

The analysis of identity and parentage, along with the capacity to diagnose disease is also of central concern to human genetic studies, particularly forensic or paternity evaluations, and in the evaluation of an individual's risk of genetic disease. Such goals have been pursued by analyzing variations in DNA sequences that distinguish the DNA of one individual from another.

If such a variation alters the lengths of the fragments that are generated by restriction endonuclease cleavage, the variations are referred to as restriction fragment length polymorphisms (“RFLPs”). RFLPs have been widely used in human genetic analyses (Glassberg, J., UK Patent Application 2135774; Skolnick, M. H. et al., Cytogen. Cell Genet. 32:58-67 (1982); Botstein, D. et al., Ann. J. Hum. Genet. 32:314-331 (1980); Fischer, S. G et al. (PCT Application WO90/13668); Uhlen, M., PCT Application WO90/11369)). Where a heritable trait can be linked to a particular RFLP, the presence of the RFLP in a target animal can be used to predict the likelihood that the animal will also exhibit the trait. Statistical methods have been developed to permit the multilocus analysis of RFLPs such that complex traits that are dependent upon multiple alleles can be mapped (Lander, S. et al., Proc. Natl. Acad. Sci. (U.S.A.) 83:7353-7357 (1986); Lander, S. et al., Proc. Natl. Acad. Sci. (U.S.A.) 84:2363-2367 (1987); Donis-Keller, H. et al., Cell 51:319-337 (1987); Lander, S. et al., Genetics 121:185-199 (1989), all herein incorporated by reference). Such methods can be used to develop a genetic map, as well as to develop animals having more desirable traits (Donis-Keller, H. et al., Cell 51:319-337 (1987); Lander, S. et al., Genetics 121:185-199 (1989)).

In some cases, the DNA sequence variations are in regions of the genome that are characterized by short tandem repeats (STRs) that include tandem di- or tri-nucleotide repeated motifs of nucleotides. These tandem repeats are also referred to as “variable number tandem repeat” (“VNTR”) polymorphisms. VNTRs have been used in identity and paternity analysis (Weber, J. L., U.S. Pat. No. 5,075,217; Armour, J. A. L. et al., FEBS Lett. 307:113-115 (1992); Jones, L. et al., Eur. J. Haematol. 39:144-147 (1987); Horn, G. T. et al., PCT Application WO91/14003; Jeffreys, A. J., European Patent Application 370,719; Jeffreys, A. J., U.S. Pat. No. 5,175,082); Jeffreys. A. J. et al., Amer. J. Hum. Genet. 39:11-24 (1986); Jeffreys. A. J. et al., Nature 316:76-79 (1985); Gray, I. C. et al., Proc. R. Acad. Soc. Lond. 243:241-253 (1991); Moore, S. S. et al., Genomics 10:654-660 (1991); Jeffreys, A. J. et al., Anim. Genet. 18:1-15 (1987); Hillel, J. et al., Anim. Genet. 20:145-155 (1989); Hillel, J. et al., Genet. 124:783-789 (1990)) and are now being used in a large number of genetic mapping studies.

A third class of DNA sequence variation results from single nucleotide polymorphisms (SNPs) that exist between individuals of the same species. Such polymorphisms are far more frequent than RFLPs, STRs and VNTRs. In some cases, such polymorphisms comprise mutations that are the determinative characteristic in a genetic disease. Indeed, such mutations may affect a single nucleotide in a protein-encoding gene in a manner sufficient to actually cause the disease (e.g. hemophilia, sickle-cell anemia, etc.). In many cases, these SNPs are in noncoding regions of a genome. Despite the central importance of such polymorphisms in modern genetics, however, no practical method has been developed that permits the use of highly parallel analysis of many SNP alleles in two or more individuals in genetic analysis.

In determining these sequence variations in human DNA samples, it is important to employ a suitable reference sequence. Such a reference sequence may include one or more of the possible target polymorphisms being examined (i.e. the reference sequence may contain one or more RFLPs, STRs and/or SNPs of interest). Alternatively, such a reference sequence may be obtained from an individual known to exhibit a particular target genetic trait, such as total red-green colorblindness, even if the specific nucleic acid sequence of the reference is not entirely known.

### SUMMARY OF THE INVENTION

The present invention is directed to a specific human genomic DNA, which is particularly useful as a reference sequence for use in genomic analysis for total red-green colorblindness. The human genomic DNA of the present invention has the following STR marker profile:

| Amel | D3S1358 | VWA | FGA | D8S1179 | D21S11 | D18S51 |
|---|---|---|---|---|---|---|
| M | 15-17 | 16-17 | 21-25 | 12-14 | 29-32.2 | 17-18 |

| D5S818 | D13S317 | D7S820 | CSF1PO | TPOX | TH01 | D16S539 |
|---|---|---|---|---|---|---|
| 11-12 | 11-12 | 8-8 | 10-11 | 8-8 | 7-9.3 | 10-13 |

This particular human genomic DNA is associated with certain genetic traits, including, but not limited to, total red-green colorblindness.

The invention also provides a method for determining the extent of genetic similarity between target DNA and the inventive DNA, which comprises the steps of: (a) determining, for a single nucleotide polymorphism of the target DNA, and for a corresponding single nucleotide polymorphism of the inventive DNA, whether the polymorphisms contain the same single nucleotide at their respective polymorphic sites; and (b) using the comparison to determine the extent of genetic similarity between the target DNA and the inventive DNA.

The invention also includes the embodiment wherein, in step (a), the determination is accomplished by a method having the sub-steps: (i) incubating a sample of nucleic acid containing the single nucleotide polymorphism of the target DNA, or the single nucleotide polymorphism of the inventive DNA, in the presence of a nucleic acid primer and at least one dideoxynucleotide derivative, under conditions sufficient to permit a polymerase mediated, template-dependent extension of the primer, the extension causing the incorporation of a single dideoxynucleotide to the 3′-terminus of the primer, the single dideoxynucleotide being complementary to the single nucleotide of the polymorphic site of the polymorphism; (ii) permitting the template-dependent extension of the primer molecule, and the incorporation of the single dideoxynucleotide; and (iii) determining the identity of the nucleotide incorporated into the polymorphic site, the identified nucleotide being complimentary to the nucleotide of the polymorphic site.

The invention further includes the embodiments of the above methods wherein the template-dependent extension of the primer is conducted in the presence of at least two dideoxynucleotide triphosphate derivatives selected from the group consisting of ddATP, ddTTP, ddCTP and ddGTP, but in the absence of dATP, dTTP, dCTP and dGTP.

The invention further includes the sub-embodiments of the above methods wherein the nucleic acid of the sample is amplified in vitro prior to the incubation, and/or the primer is immobilized to a solid support.

The invention further concerns the embodiment of the above methods wherein a non-invasive swab is used to collect the sample of target DNA.

The invention further provides a method for predicting whether a cell containing target DNA will exhibit a predetermined trait associated with the reference DNA which comprises the steps: (a) identifying one or more alleles associated with the trait, each allele being a single nucleotide polymorphic allele having a single nucleotide polymorphic site; (b) determining for each of the single nucleotide polymorphic alleles, a nucleotide present at the allele's polymorphic site in the reference DNA, to thereby define a set of single nucleotides at a set of polymorphic sites that are present in a human either exhibiting the trait or not exhibiting the trait, as desired; (c) determining the identity of single nucleotides present at corresponding single nucleotide polymorphic alleles of the target DNA; and (d) comparing the identity of the single nucleotides present at the polymorphic sites of the polymorphisms of the inventive DNA with the single nucleotides present at the corresponding single nucleotide polymorphic alleles of the target DNA.

### BRIEF DESCRIPTION OF THE FIGURES

FIG. 1 illustrates the preferred method for cloning random genomic fragments. Genomic DNA is size fractionated, and then introduced into a plasmid vector, in order to obtain random clones. PCR primers are designed, and used to sequence the inserted genomic sequences.

FIG. 2 shows a graph of the probability that two individuals will have identical genotypes with given panels of genetic markers. The number of tests employed is plotted on the abscissa while the cumulative probability of non-identity is plotted on the ordinate. The horizontal line indicates 0.95 probability of non-identity. Legend: o indicates the extrapolated prototype; x indicates 3 alleles (51%, 34%, 15%); triangle indicates 2 alleles (79%, 21%).

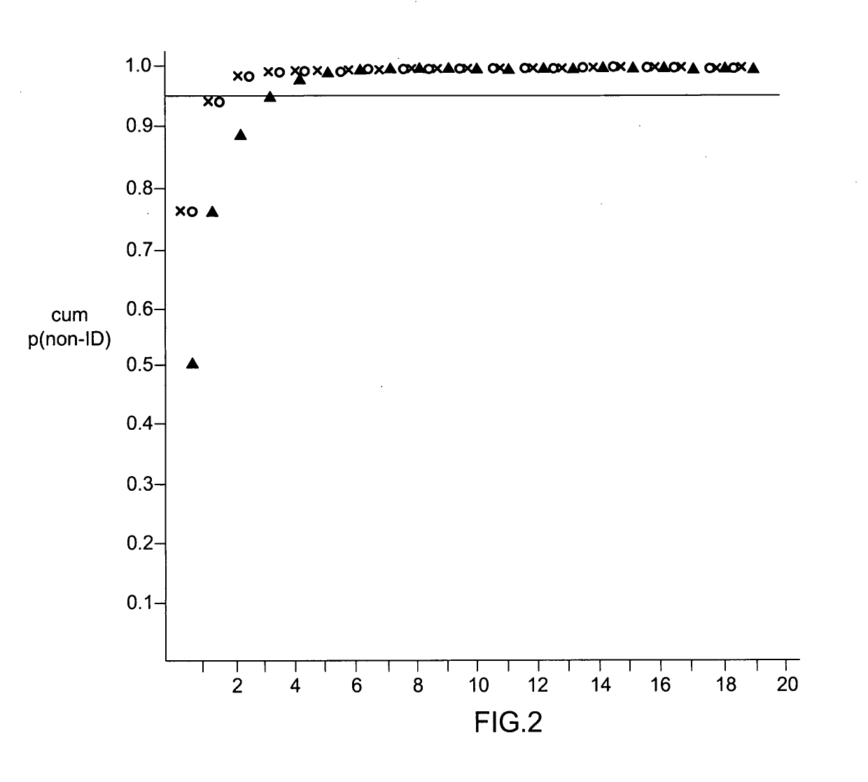

### DESCRIPTION OF THE PREFERRED EMBODIMENTS

### A. Reference DNA

The total sequence of human genomic DNA is now generally known and widely avialable to the interested public (for maps of the human genomic DNA and its component sequences, see, for example, www.ncbi.nlm.nih.gov/projects/genome/genemap99; and www.ornl.gov/sci/techresources/Human_Genome/posters/chromosome; and their associated inks). Within this known sequence, however, there exist a number of possible variations (as discussed above). A specific combination of particular variations constitutes a simple, basic and fundamental representation of the genomic DNA of a unique human being. For example, a specific combination of fourteen (14) STR markers in a male caucasian is believed to be occur only once in 253 trillion possibilities, and is therefore unique.

The DNA of the present invention is an isolated and/or purified sample of a human genomic DNA which is associated with certain genetic traits, such as total red-green colorblindness. The particular human genomic DNA of the present invention has the following STR marker profile:

| Amel | D3S1358 | VWA | FGA | D8S1179 | D21S11 | D18S51 |
|---|---|---|---|---|---|---|
| M | 15-17 | 16-17 | 21-25 | 12-14 | 29-32.2 | 17-18 |

| D5S818 | D13S317 | D7S820 | CSF1PO | TPOX | TH01 | D16S539 |
|---|---|---|---|---|---|---|
| 11-12 | 11-12 | 8-8 | 10-11 | 8-8 | 7-9.3 | 10-13 |

The inventive human genomic DNA is useful as a reference for studying variations in the human genome that may be associated with certain genetic traits or diseases. The inventive human genomic DNA is particularly useful as a positive reference for studying target genomes for polymorphisms and the like which may be associated with total red-green colorblindness.

### B. Genetic Identification Using Polymorphisms

Particular gene sequences of interest for study using the present invention include “single nucleotide polymorphisms” (or “SNPs”). A “polymorphism” is a variation in the DNA sequence of some members of a species. The genomes of animals naturally undergo spontaneous mutation in the course of their continuing evolution (Gusella, J. F., Ann. Rev. Biochem. 55:831-854 (1986)). The majority of such mutations create polymorphisms. The mutated sequence and the initial sequence co-exist in the species' population. In some instances, such co-existence is in stable or quasi-stable equilibrium. In other instances, the mutation confers a survival or evolutionary advantage to the species, and accordingly, it may eventually (i.e. over evolutionary time) be incorporated into the DNA of every member of that species.

A polymorphism is thus said to be “allelic,” in that, due to the existence of the polymorphism, some members of a species may have the unmutated sequence (i.e. the original “allele”) whereas other members may have a mutated sequence (i.e. the variant or mutant “allele”). In the simplest case, only one mutated sequence may exist, and the polymorphism is said to be cliallelic. Diallelic polymorphisms are the most common and the preferred polymorphisms of the present invention. The occurrence of alternative mutations can give rise to trialleleic, etc. polymorphisms. An allele may be referred to by the nucleotide(s) that comprise the mutation.

Aspects of the present invention involve the use of the polymorphisms on the inventive DNA in genotyping a human. Such allelic polymorphisms are referred to herein as “single nucleotide polymorphisms,” or “SNPs.” “Single nucleotide polymorphisms” are defined by the following attributes. A central attribute of such a polymorphism is that it contains a polymorphic site, “X,” most preferably occupied by a single nucleotide, which is the site of variation between allelic sequences. A second characteristic of an SNP is that its polymorphic site “X” is preferably preceded by and followed by “invariant” sequences of the allele. The polymorphic site of the SNP is thus said to lie “immediately” 3′ to a “5′-proximal” invariant sequence, and “immediately” 5′ to a “3′-distal” invariant sequence. Such sequences flank the polymorphic site.

As used herein, a sequence is said to be an “invariant” sequence of an allele if the sequence does not vary in the population, and if mapped, would map to a “corresponding” sequence of the same allele in the genome of every member of the population. Two sequences are said to be “corresponding” sequences if they are analogs of one another obtained from different sources. The gene sequences that encode hemoglobin in two humans illustrate “corresponding” allelic sequences. The definition of “corresponding alleles” provided herein is intended to clarify, but not to alter, the meaning of that term as understood by those of ordinary skill in the art.

Since genomic DNA is double-stranded, each SNP can be defined in terms of either strand. Thus, for every SNP, one strand will contain an immediately 5′-proximal invariant sequence and the other will contain an immediately 3′-distal invariant sequence. In the preferred embodiment, wherein a SNP's polymorphic site, “X,” is a single nucleotide, each strand of the double-stranded DNA of the SNP will contain both an immediately 5′-proximal invariant sequence and an immediately 3′-distal invariant sequence.

Although many of the SNPs identified using the methods of the present invention involve a substitution of one nucleotide for another at the SNP's polymorphic site, SNPs can also be more complex, and may comprise a deletion of a nucleotide from, or an insertion of a nucleotide into, one of two corresponding sequences. For example, a particular gene sequence may contain an A in a particular polymorphic site in some, whereas in others a single or multiple base deletion might be present at that site. Although the preferred SNPs used in the methods of the present invention have both an invariant proximal sequence and invariant distal sequence, SNPs may have only an invariant proximal or only an invariant distal sequence.

Nucleic acid molecules having the a sequence complementary to that of an immediately 3′-distal invariant sequence of a SNP can, if extended in a “template-dependent” manner, form an extension product that would contain the SNP's polymorphic site. A preferred example of such a nucleic acid molecule is a nucleic acid molecule whose sequence is the same as that of a 5′-proximal invariant sequence of the SNP. “Template-dependent” extension refers to the capacity of a polymerase to mediate the extension of a primer such that the extended sequence is complementary to the sequence of a nucleic acid template. A “primer” is a single-stranded oligonucleotide or a single-stranded polynucleotide that is capable of being extended by the covalent addition of a nucleotide in a “template-dependent” extension reaction. In order to possess such a capability, the primer must have a 3′-hydroxyl terminus, and be hybridized to a second nucleic acid molecule (i.e. the “template”). A primer is typically 11 bases or longer; most preferably, a primer is 20 bases, however, primers of shorter or greater length may suffice. A “polymerase” is an enzyme that is capable of incorporating nucleoside triphosphates to extend a 3′-hydroxyl group of a nucleic acid molecule, if that molecule has hybridized to a suitable template nucleic acid molecule. Polymerase enzymes are discussed in Watson, J. D., In: Molecular Biology of the Gene, 3rd Ed., W. A. Benjamin, Inc., Menlo Park, Calif. (1977), which reference is incorporated herein by reference, and similar texts. Other polymerases such as the large proteolytic fragment of the DNA polymerase I of the bacterium E. coli, commonly known as “Klenow” polymerase, E. coli DNA polymerase I, and bacteriophage T7 DNA polymerase, may also be used to perform the method described herein. Nucleic acids having the same sequence as that of the immediately 3′ distal invariant sequence of a SNP can be ligated in a template dependent fashion to a primer that has the same sequence as that of the immediately 5′ proximal sequence that has been extended by one nucleotide in a template dependent fashion.

The single nucleotide polymorphic sites can be used to analyze the DNA of any human. SNPs have several salient advantages over RFLPs, STRs and VNTRs, although these techniques may also be employed when using the inventive DNA as a reference.

First, SNPs occur at greater frequency (approximately 10-100 fold greater), and with greater uniformity than RFLPs and VNTRs. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g. predisposition to cancer) reflects a mutation at a particular locus, then any polymorphism that is linked to the particular locus can be used to predict the probability that an individual will be exhibiting that trait.

The value of such a prediction is determined in part by the distance between the polymorphism and the locus. Thus, if the locus is located far from any repeated tandem nucleotide sequence motifs, VNTR analysis will be of very limited value. Similarly, if the locus is far from any detectable RFLP, an RFLP analysis would not be accurate. However, since the SNPs of the present invention are present approximately once every 300 bases in the mammalian genome, and exhibit uniformity of distribution, a SNP can, statistically, be found within 150 bases of any particular genetic lesion or mutation. Indeed, the particular mutation may itself be an SNP. Thus, where such locus has been sequenced, the variation in that locus' nucleotide is determinative of the trait in question.

Second, SNPs are more stable than other classes of polymorphisms. Their spontaneous mutation rate is approximately 10⁻⁹, approximately 1,000 times less frequent than VNTRs. Significantly, VNTR-type polymorphisms are characterized by high mutation rates.

Third, SNPs have the further advantage that their allelic frequency can be inferred from the study of relatively few representative samples. These attributes of SNPs permit a much higher degree of genetic resolution of identity, paternity exclusion, and analysis of an animal's predisposition for a particular genetic trait than is possible with either RFLP or VNTR polymorphisms.

Fourth, SNPs reflect the highest possible definition of genetic information—nucleotide position and base identity. Despite providing such a high degree of definition, SNPs can be detected more readily than either RFLPs or VNTRs, and with greater flexibility. Indeed, because DNA is double-stranded, the complimentary strand of the allele can be analyzed to confirm the presence and identity of any SNP.

The flexibility with which an identified SNP can be characterized is a salient feature of SNPs. VNTR-type polymorphisms, for example, are most easily detected through size fractionation methods that can discern a variation in the number of the repeats. RFLPs are most easily detected by size fractionation methods following restriction digestion.

In contrast, SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation.

The “Genetic Bit Analysis (“GBA”) method disclosed by Goelet, P. et al. (WO 92/15712, herein incorporated by reference), is a preferred method for detecting the single nucleotide polymorphisms of the present invention. GBA is a method of polymorphic site interrogation in which the nucleotide sequence information surrounding the site of variation in a target DNA sequence is used to design an oligonucleotide primer that is complementary to the region immediately adjacent to, but not including, the variable nucleotide in the target DNA. The target DNA template is selected from the biological sample and hybridized to the interrogating primer. This primer is extended by a single labeled dideoxynucleotide using DNA polymerase in the presence of two, and preferably all four chain terminating nucleoside triphosphate precursors. Cohen, D. et al. (PCT Application WO91/02087) describes a related method of genotyping.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvnen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyrn, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvnen, A.-C., et al., Amer. J. Hum. Genet. 52:46-59 (1993)). Such a range of locus-specific signals could be more complex to interpret, especially for heterozygotes, compared to the simple, ternary (2:0, 1:1, or 0:2) class of signals produced by the GBA method. In addition, for some loci, incorporation of an incorrect deoxynucleotide can occur even in the presence of the correct dideoxynucleotide (Komher, J. S. et al., Nucl. Acids. Res. 17:7779-7784 (1989)). Such deoxynucleotide misincorporation events may be due to the Km of the DNA polymerase for the mispaired deoxy-substrate being comparable, in some sequence contexts, to the relatively poor Km of even a correctly base paired dideoxy-substrate (Kornberg, A., et al., In: DNA Replication, 2nd Edition, W. H. Freeman and Co., (1992); New York; Tabor, S. et al., Proc. Natl. Acad. Sci. (U.S.A.) 86:4076-4080 (1989)). This effect would contribute to the background noise in the polymorphic site interrogation.

### C. Methods for Discovering Novel Polymorphic Sites

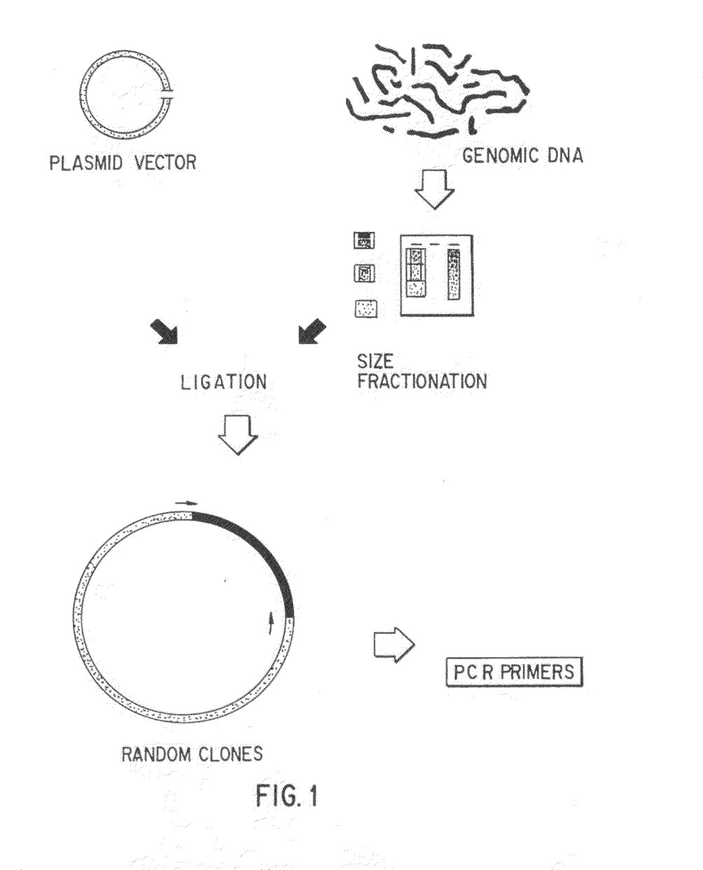

A preferred method for discovering polymorphic sites involves comparative sequencing of genomic DNA fragments from a number of haploid genomes and comparing those sequences to a corresponding fragment of the inventive DNA. In the preferred embodiment, illustrated in FIG. 1, such sequencing is performed by preparing a random genomic library that contains 0.5-3 kb fragments of the inventive DNA. Sequences of these recombinants are then used to facilitate PCR sequencing of a number of randomly selected individuals at the same genomic loci.

From such genomic libraries (typically of approximately 50,000 clones), several hundred (200-500) individual clones are purified, and the sequences of the termini of their inserts are determined. Only a small amount of terminal sequence data (100-200 bases) need be obtained to permit PCR amplification of the cloned region. The purpose of the sequencing is to obtain enough sequence information to permit the synthesis of primers suitable for mediating the amplification of the equivalent fragments from genomic DNA samples of other target members. Preferably, such sequence determinations are performed using cycle sequencing methodology.

The primers are used to amplify DNA from a panel of selected target individuals. The number of individuals in the panel determines the lowest frequency of the polymorphisms that are to be isolated. Thus, if six members are evaluated, a polymorphism that exists at a frequency of, for example, 0.01 might not be identified. In an illustrative, but oversimplified, mathematical treatment, a sampling of six members would be expected to identify only those polymorphisms that occur at a frequency of greater than about 0.08 (i.e. 1.0 total frequency divided by 6 members divided by 2 alleles per genome).

Thus, if one desires the identification of less frequent polymorphisms, a greater number of panel members must be evaluated.

Cycle sequence analysis (Mullis, K. et al., Cold Spring Harbor Symp. Quant. Biol. 51:263-273 (1986); Erlich H. et al., European Patent Appln. 50,424; European Patent Appln. 84,796, European Patent Application 258,017, European Patent Appln. 237,362;

Mullis, K., European Patent Appln. 201,184; Mullis K. et al., U.S. Pat. No. 4,683,202; Erlich, H., U.S. Pat. No. 4,582,788; and Saiki, R. et al., U.S. Pat. No. 4,683,194)) is facilitated through the use of automated DNA sequencing instruments and software (Applied Biosystems, Inc.). Differences between sequences of different animals can thereby be identified and confirmed by inspecting the relevant portion of the chromatograms on the computer screen. Differences are interpreted to reflect a DNA polymorphism only if the data was available for both strands, and present in more than one haploid example among the population of animals tested.

Despite the randomized nature of such a search for polymorphisms, such sequencing and comparison of random DNA clones is readily able to identify suitable polymorphisms.

The restriction digestion patterns obtained from the genomic DNAs are preferably compared directly to the patterns obtained from PCR products generated using the corresponding plasmid templates. Such a comparison provides an internal control which indicates that the amplified sequences from the genomic and plasmid DNAs derive from equivalent loci. This control also allows identification of primers that fortuitously amplify repeated sequences, or multicopy loci, since these will generate many more fragments from the genomic DNA templates than from the plasmid templates.

### D. Methods for Genotyping Single Nucleotide Polymorphisms

Any of a variety of methods can be used to identify the polymorphic site, “X,” of a single nucleotide polymorphism of a target DNA. The preferred method of such identification involves directly ascertaining the sequence of the polymorphic site for each polymorphism being analyzed. This approach is thus markedly different from the RFLP method which analyzes patterns of bands rather than the specific sequence of a polymorphism.

Nucleic acid specimens may be obtained from an individual of the species that is to be analyzed using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include blood collection, semen collection, needle biopsy, pleural aspiration, etc. Examples of such methods are discussed by Kim, C. H. et al. (J. Virol. 66:3879-3882 (1992)); Biswas, B. et al. (Annals NY Acad. Sci. 590:582-583 (1990)); Biswas, B. et al. (J. Clin. Microbiol. 29:2228-2233 (1991)).

In contrast, a “non-invasive” sampling means is one in which the nucleic acid molecules are recovered from an internal or external surface of the animal. Examples of such “non-invasive” sampling means include “swabbing,” collection of tears, saliva, urine, fecal material, sweat or perspiration, etc. As used herein, “swabbing” denotes contacting an applicator/collector (“swab”) containing or comprising an adsorbent material to a surface in a manner sufficient to collect surface debris and/or dead or sloughed off cells or cellular debris. Such collection may be accomplished by swabbing nasal, oral, rectal, vaginal or aural orifices, by contacting the skin or tear ducts, by collecting hair follicles, etc.

Nasal swabs have been used to obtain clinical specimens for PCR amplification (Olive, D. M. et al., J. Gen. Virol. 71:2141-2147 (1990); Wheeler, J. G. et al., Amer. J. Vet. Res. 52:1799-1803 (1991)). The use of hair follicles to identify VNTR polymorphisms for paternity testing in horses has been described by Ellegren, H. et al. (Animal Genetics 23:133-142 (1992). The reference states that a standardized testing system based on PCR-analyzed microsatellite polymorphisms are likely to be an alternative to blood typing for paternity testing.

A preferred swab for the collection of DNA will comprise a solid support, at least a portion of which is designed to adsorb DNA. The portion designed to adsorb. DNA may be of a compressible texture, such as a “foam rubber,” or the like. Alternatively, it may be an adsorptive fibrous composition, such as cotton, polyester, nylon, or the like. In yet another embodiment, the portion designed to adsorb DNA may be an abrasive material, such as a bristle or brush, or having a rough surface. The portion of the swab that is designed to adsorb DNA may be a combination of the above textures and compositions (such as a compressible brush, etc.). The swab will, preferably, be specially formed in a substantially rod-like, arrow-like or mushroom-like shape, such that it will have a segment that can be held by the collecting individual, and a tip or end portion which can be placed into contact with the surface that contains the sample DNA that is to be collected. In one embodiment, the swab will be provided with a storage chamber, such as a plastic or glass tube or cylinder, which may have one open end, such as a test-tube. Alternatively, the tube may have two open ends, such that after swabbing, the collector can pull on one end of the swab so as to cause the other end of the swab to be withdrawn into the tube. In yet another embodiment, the tube may have two open ends, such that after swabbing, the tube can be converted into a column to assist in the further processing of the collected DNA. In one embodiment, the end or ends of the storage chamber are self-sealing after swabbing has been accomplished.

The swab or the storage chamber may contain antimicrobial agents at concentrations sufficient to prevent the proliferation of microbes (bacteria, yeast, molds, etc.) during subsequent storage or handling.

In one embodiment, the swab or storage chamber will contain an chromogenic reagent which reacts to the presence of DNA to yield a detectable signal that can be identified at the time of sample collection. Most preferably, such a reagent will comprise a minimum concentration “open-end point” assay for DNA. Such an assay is capable of detecting concentrations of nucleic acids that range from the minimum detection level of the assay to the maximum assay saturation level of the assay. This saturation level is adjustable, and can be increased by decreasing the time of reaction. Preferred chromogenic reagents include anti-DNA antibodies that are conjugated to enzymes, diaminopimelic acid, etc.

The detection of polymorphic sites in a sample of DNA may be facilitated through the use of DNA amplification methods. Such methods specifically increase the concentration of sequences that span the polymorphic site, or include that site and sequences located either distal or proximal to it. Such amplified molecules can be readily detected by gel electrophoresis or other means.

The most preferred method of achieving such amplification employs PCR, using primer pairs that are capable of hybridizing to the proximal sequences that define a polymorphism in its double-stranded form.

In lieu of PCR, alternative methods, such as the “Ligase Chain Reaction” (“LCR”) may be used (Barany, F., Proc. Natl. Acad. Sci. (U.S.A.) 88:189-193 (1991). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

In accordance with the present invention, LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide.

In an alternative embodiment, the oligonucleotides will not include the polymorphic site, such that when they hybridize to the target molecule, a “gap” is created (see, Segev, D., PCT Application WO 90/01069). This gap is then “filed” with complementary dNTPs (as mediated by DNA polymerase), or by an additional pair of oligonucleotides. Thus, at the end of each cycle, each single strand has a complement capable of serving as a target during the next cycle and exponential amplification of the desired sequence is obtained.

The “Oligonucleotide Ligation Assay” (“OLA”) (Landegren, U. et al., Science 241:1077-1080 (1988)) shares certain similarities with LCR and may also be adapted for use in polymorphic analysis.

The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonucleotide, are also known (Wu, D. Y. et al., Genomics 4:560 (1989)), and may be readily adapted to the purposes of the present invention.

Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT appln. WO 89/06700; Kwoh, D. et al., Proc. Natl. Acad. Sci. (U.S.A.) 86:1173 (1989); Gingeras, T. R. et al., PCT application WO 88/10315)), or isothermal amplification methods (Walker, G. T. et al., Proc. Natl. Acad. Sci. (U.S.A.) 89:392-396 (1992)) may also be used.

The direct analysis of the sequence of an SNP can be accomplished using either the “dideoxy-mediated chain termination method,” also known as the “Sanger Method” (Sanger, F., et al., J. Molec. Biol. 94:441 (1975)) or the “chemical degradation method,” “also known as the “Maxam-Gilbert method” (Maxam, A. M., et al., Proc. Nad. Acad. Sci. (U.S.A.) 74:560 (1977), both references herein incorporated by reference). Methods for sequencing DNA using either the dideoxy-mediated method or the Maxam-Gilbert method are widely known to those of ordinary skill in the art. Such methods are, for example, disclosed in Sambrook, J. et al., Molecular Cloning, a Laboratory Manual, 2nd Edition. Cold Spring Harbor Press, Cold Spring Harbor, N.Y. (1989), and in Zyskind, J. W., et al., Recombinant DNA Laboratory Manual, Academic Press. Inc., New York (1988), both herein incorporated by reference.

Where a nucleic acid sample contains double-stranded DNA (or RNA), or where a double-stranded nucleic acid amplification protocol (such as PCR) has been employed, it is generally desirable to conduct such sequence analysis after treating the double-stranded molecules so as to obtain a preparation that is enriched for, and preferably predominantly, only one of the two strands.

The simplest method for generating single-stranded DNA molecules from double-stranded DNA is denaturation using heat or alkalai treatment.

Single-stranded DNA molecules may also be produced using the single-stranded DNA bacteriophage M13 (Messing, J. et al., Meth. Enzymol. 101:20 (1983); see also, Sambrook, J., et al. (In: Molecular Cloning: A Laboratory Manual, Cold Spring Harbor Laboratory Press, Cold Spring Harbor, N.Y. (1989)).

Several alternative methods can be used to generate single-stranded DNA molecules. Gyllensten, U. et al., (Proc. Nad. Acad. Sci. (U.S.A.) 85:7652-7656 (1988) and Mihovilovic, M. et al., (BioTechniques 7(1):14 (1989)) describe a method, termed “asymmetric PCR,” in which the standard “PCR” method is conducted using primers that are present in different molar concentrations.

Higuchi, R. G. et al. (Nucleic Acids Res. 17:5865 (1985)) exemplifies an additional method for generating single-stranded amplification products. The method entails phosphorylating the 5′-terminus of one strand of a double-stranded amplification product, and then permitting a 5′-3′ exonuclease (such as exonuclease) to preferentially degrade the phosphorylated strand.

Other methods have also exploited the nuclease resistant properties of phosphorothioate derivatives in order to generate single-stranded DNA molecules (Benkovic et al., U.S. Pat. No. 4,521,509; Jun. 4, 1985); Sayers, J. R. et al. (Nucl. Acids Res. 16:791-802 (1988); Eckstein, F. et al., Biochemistry 15:1685-1691 (1976); Ott, J. et al., Biochemistry 26:8237-8241 (1987)).

A discussion of the relative advantages and disadvantages of such methods of producing single-stranded molecules is provided by Nikiforov, T. (U.S. patent application Ser. No. 08/005,061, herein incorporated by reference).

Most preferably, such single-stranded molecules will be produced using the methods described by Nikiforov, T. (U.S. patent application Ser. No. 08/005,061, herein incorporated by reference). In brief, these methods employ nuclease resistant nucleotides derivatives, and incorporates such derivatives, by chemical synthesis or enzymatic means, into primer molecules, or their extension products, in place of naturally occurring nucleotides.

Suitable nucleotide derivatives include derivatives in which one or two of the non-bridging oxygens of the phosphate moiety of a nucleotide has been replaced with a sulfur-containing group (especially a phosphorothioate), an alkyl group (especially a methyl or ethyl alkyl group), a nitrogen-containing group (especially an amine), and/or a selenium-containing group, etc.

Phosphorothioate deoxyribonucleotide or ribonucleotide derivatives (e.g. a nucleoside 5′-O-1-thiotriphosphate) are the most preferred nucleotide, derivatives. Any of a variety of chemical methods may be used to produce such phosphorothioate derivatives (see, for example, Zon, G. et al., Anti-Canc. Drug Des. 6:539-568 (1991); Kim, S. G. et al., Biochem. Biophys. Res. Commun. 179:1614-1619 (1991); Vu, H. et al., Tetrahedron Lett. 32:3005-3008 (1991); Taylor, J. W. et al., Nucl. Acids Res. 13:8749-8764 (1985); Eckstein, F. et al., Biochemistry 15:1685-1691 (1976); Ott, J. et al., Biochemistry 26:8237-8241 (1987); Ludwig; J. et al., J. Ora. Chem. 54:631-635 (1989), all herein incorporated by reference). Phosphorothioate nucleotide derivatives can also be obtained commercially from Amersham or Pharmacia.

Importantly, the selected nucleotide derivative must be suitable for in vitro primer-mediated extension and provide nuclease resistance to the region of the nucleic acid molecule in which it is incorporated. In the most preferred embodiment, it must confer resistance to exonucleases that attack double-stranded DNA from the 5′-end (5′-3′ exonucleases). Examples of such exonucleases include bacteriophage T7 gene 6 exonuclease (“T7 exonuclease) and the bacteriophage lambda exonuclease (“.lambda. exonuclease”). Both T7 exonuclease and .lambda. exonuclease are inhibited to a significant degree by the presence of phosphorothioate bonds so as to allow the selective degradation of one of the strands. However, any double-strand specific, 5′-3′ exonuclease can be used for this process, provided that its activity is affected by the presence of the bonds of the nuclease resistant nucleotide derivatives. The preferred enzyme when using phosphorothioate derivatives is the 17 gene 6 exonuclease, which shows maximal enzymatic activity in the same buffer used for many DNA dependent polymerase buffers including Taq polymerase. The 5′-3′ exonuclease resistant properties of phosphorothioate derivative-containing DNA molecules are discussed, for example, in Kunkel, T. A. (In: Nucleic Acids and Molecular Biology, Vol. 2, 124-135 (Eckstein, F. et al., eds.), Springer-Verlag, Berlin, (1988)). The 3′-5′ exonuclease resistant properties of phosphorothioate nucleotide containing nucleic acid molecules are disclosed in Putney, S. D., et al. (Proc. Natl. Acad. Sci. (U.S.A.) 78:7350-7354 (1981)) and Gupta, A. P., et al. (Nucl. Acids. Res., 12:5897-5911 (1984)).

In addition to being resistant to such exonucleases, nucleic acid molecules that contain phosphorothioate derivatives at restriction endonuclease cleavage recognition sites are resistant to such cleavage. Taylor, J. W., et al. (Nucl. Acids Res., 13:8749-8764 (1985)) discusses the endonuclease resistant properties of phosphorothioate nucleotide containing nucleic acid molecules.

The nuclease resistance of phosphorothioate bonds has been utilized in a DNA amplification protocol (Walker, T. G. et al. (Proc. Nad. Acad. Sci. (U.S.A.) 89:392-396 (1992)). In the Walker et al. method, phosphorothioate nucleotide derivatives are installed within a restriction endonuclease recognition site in one strand of a double-stranded DNA molecule. The presence of the phosphorothioate nucleotide derivatives protects that strand from cleavage, and thus results in the nicking of the unprotected strand by the restriction endonuclease. Amplification is accomplished by cycling the nicking and polymerization of the strands.

Similarly, this resistance to nuclease attack has been used as the basis for a modified “Sanger” sequencing method (Labeit, S. et al. (DNA 5:173-177 (1986)). In the Labeit et al. method, S-labeled phosphorothioate nucleotide derivatives were employed in lieu of the dideoxy nucleotides of the “Sanger” method.

In the most preferred embodiment, the phosphorothioate derivative is included in the primer. The nucleotide derivative may be incorporated into any position of the primer, but will preferably be incorporated at the 5′-terminus of the primer, most preferably adjacent to one another. Preferably, the primer molecules will be approximately 25 nucleotides in length, and contain from about 4% to about 100%, and more preferably from about 4% to about 40%, and most preferably about 16%, phosphorothioate residues (as compared to total residues). The nucleotides may be incorporated into any position of the primer, and may be adjacent to one another, or interspersed across all or part of the primer.

In one embodiment, the present invention can be used in concert with an amplification protocol, for example, PCR. In this embodiment, it is preferred to limit the number of phosphorothioate bonds of the primers to about 10 (or approximately half of the length of the primers), so that the primers can be used in a PCR reaction without any changes to the PCR protocol that has been established for non-modified primers. When the primers contain more phosphorothioate bonds, the PCR conditions may require adjustment, especially of the annealing temperature, in order to optimize the reaction.

The incorporation of such nucleotide derivatives into DNA or RNA can be accomplished enzymatically, using a DNA polymerase (Vosberg, H. P. et al., Biochemistry 16: 3633-3640 (1977); Burgers, P. M. J. et al., J. Biol. Chem. 254:6889-6893 (1979); Kunkel, T. A., In: Nucleic Acids and Molecular Biology, Vol 2, 124-135 (Eckstein, F. et al., eds.), Springer-Verlag, Berlin, (1988); Olsen, D. B. et al., Proc. Natl. Acad. Sci. (U.S.A.) 87:1451-1455 (1990); Griep, M. A. et al., Biochemistry 29:9006-9014 (1990); Sayers, J. R. et al., Nucl. Acids Res. 16:791-802 (1988)). Alternatively, phosphorothioate nucleotide derivatives can be incorporated synthetically into an oligonucleotide (Zon, G. et al., Anti-Canc. Drug Des. 6:539-568 (1991)).

The primer molecules are permitted to hybridize to a complementary target nucleic acid molecule, and are then extended, preferably via a polymerase, to form an extension product. The presence of the phosphorothioate nucleotides in the primers renders the extension product resistant to nuclease attack. As indicated, the amplification products containing phosphorothioate or other suitable nucleotide derivatives are substantially resistant to “elimination” (i.e. degradation) by “5′-3′” exonucleases such as T7 exonuclease or exonuclease, and thus a 5′-3′ exonuclease will be substantially incapable of further degrading a nucleic acid molecule once it has encountered a phosphorothioate residue.

Since the target molecule lacks nuclease resistant residues, the incubation of the extension product and its template—the target—in the presence of a 5′-3′ exonuclease results in the destruction of the template strand, and thereby achieves the preferential production of the desired single strand.

### E. Solid Phase Attachment of DNA

The preferred method of determining the identity of the polymorphic site of a polymorphism involves nucleic acid hybridization. Although such hybridization can be performed in solution (Berk, A. J., et al. Cell 12:721-732 (1977); Hood, L. E., et al., In: Molecular Biology of Eukaryotic Cells: A Problems Approach, Menlo Park, Calif.: Benjamin-Cummings, (1975); Wetmer, J. G., Hybridization and Renaturation Kinetics of Nucleic Acids. Ann. Rev. Biophys. Bioeng. 5:337-361 (1976); Itakura, K., et al., Ann. Rev. Biochem. 53:323-356, (1984)), it is preferable to employ a solid-phase hybridization assay (see, Saiki, R. K. et al. Proc. Natl. Acad. Sci. (U.S.A.) 86:6230-6234 (1989); Gilham et al., J. Amer. Chem. Soc. 86:4982 (1964) and Kremsky et al., Nucl. Acids Res. 15:3131-3139 (1987)).

Any of a variety of methods can be used to immobilize oligonucleotides to the solid support. One of the most widely used methods to achieve such an immobilization of oligonucleotide primers for subsequent use in hybridization-based assays consists of the non-covalent coating of these solid phases with streptavidin or avidin and the subsequent immobilization of biotinylated oligonucleotides (Holmstrom, K. et al., Anal. Biochem. 209:278-283 (1993)). Another known method (Running. J. A. et al., BioTechniques 8:276-277 (1990); Newton, C. R. et al. Nud. Acids Res. 21:1155-1162 (1993)) requires the pre-coating of the polystyrene or glass solid phases with poly-L-Lys or poly L-Lys, Phe, followed by the covalent attachment of either amino- or sulfhydryl-modified oligonucleotides using bifunctional crosslinking reagents. Both methods have the disadvantage of requiring the use of modified oligonucleotides as well as a pre-treatment of the solid phase.

In another published method (Kawai, S et al., Anal. Biochem. 209:63-69 (1993)), short oligonucleotide probes were ligated together to form multimers and these were ligated into a phagemid vector. Following in vitro amplification and isolation of the single-stranded form of these phagemids, they were immobilized onto polystyrene plates and fixed by UV irradiation at 254 nm. The probes immobilized in this way were then used to capture and detect a biotinylated PCR product.

A method for the direct covalent attachment of short, 5′-phosphorylated primers to chemically modified polystyrene plates (“Covalink” plates, Nunc) has also been published (Rasmussen, S. R. et al., Anal. Biochem. 198:138-142 (1991)). The covalent bond between the modified oligonucleotide and the solid phase surface is introduced by condensation with a water-soluble carbodiimide. This method is claimed to assure a predominantly 5′-attachment of the oligonucleotides via their 5′-phosphates; however, it requires the use of specially prepared, expensive plates.

Most preferably, such immobilization of oligonucleotides (preferably between 15 and 30 bases) is accomplished using a method that can be used directly, without the need for any pre-treatment of commercially available polystyrene microwell plates (ELISA plates) or microscope glass slides. Since 96 well polystyrene plates are widely used in ELISA tests, there has been significant interest in the development of methods for the immobilization of short oligonucleotide primers to the wells of these plates for subsequent hybridization assays. Also of interest is a method for the immobilization to microscope glass slides, since the latter are used in the so-called Slide Immunoenzymatic Assay (SIA) (de Macario, E. C. et al., BioTechniques 3:138-145 (1985)).

The solid support can be glass, plastic, paper, etc. The support can be fashioned as a bead, dipstick, test tube, etc. In a preferred embodiment, the support will be a microtiter dish, having a multiplicity of wells. The conventional 96-well microliter dishes used in diagnostic laboratories and in tissue culture are a preferred support. The use of such a support allows the simultaneous determination of a large number of samples and controls, and thus facilitates the analysis. Automated delivery systems can be used to provide reagents to such microtiter dishes. Similarly, spectrophotometric methods can be used to analyze the polymorphic sites, and such analysis can be conducted using automated spectrophotometers.

According to one method for immobilizing oligonucleotides for such analysis, any of a number of commercially available polystyrene plates can be used directly for the immobilization, provided that they have a hydrophilic surface. Examples of suitable plates include the Immulon 4 plates (Dynatech) and the Maxisorp plates (Nunc). The immobilization of the oligonucleotides to the plates is achieved simply by incubation in the presence of a suitable salt. No immobilization takes place in the absence of a salt, i.e., when the oligonucleotide is present in a water solution. Examples for suitable salts are: 50-250 mM NaCl; 30-100 mM 1-ethyl-3-(3′-dimethylaminopropyl)carbodiimide hydrochloride (EDC), pH 6.8; 50-150 mM octyldimethylamine hydrochloride, pH 7.0; 50-250 mM tetramethylammonium chloride. The immobilization is achieved by incubation, preferably at room temperature or 3 to 24 hours. After such incubation, the plates are washed, preferably with a solution of 10 mM Tris HCl, pH 7.5, containing 150 mM NaCl and 0.05% vol. Tween-20 (TNTw). The latter ingredient serves the important role of blocking all free oligonucleotide binding sites still present on the polystyrene surface, so that no nonspecific binding of oligonucleotides can take place during the subsequent hybridization steps. Using radioactively labeled oligonucleotides, the amount of immobilized oligonucleotides per well was determined to be at least 500 fmoles. The oligonucleotides are immobilized to the surface of the plate with sufficient stability and can only be removed by prolonged incubations with 0.5 M NaOH solutions at elevated temperatures. No oligonucleotide is removed by washing the plate with water, TNTw (Tween 20), PBS, 1.5 M NaCl, or other similar solutions.

The immobilized oligonucleotides can be used to capture specific DNA sequences by hybridization. The hybridization is usually carried out in a solution containing 1.5 M NaCl and 10 mM EDTA, for 15 to 30 minutes at room temperature. Other hybridization conditions can also be used. More than 400 fmoles of a specific DNA sequence was found to hybridize to the immobilized oligonucleotide in one well. This DNA is bound to the initially immobilized oligonucleotide only via Watson-Crick hydrogen bonds can be easily removed from the wells by a brief wash with a 0.1 M NaOH solution, without removing the initially attached oligonucleotide from the plate. If the captured DNA fragment is nonradioactively labeled, e.g., with a biotin residue, the detection can be carried out using a suitable enzyme-linked assay.

Although no modifications have to be introduced into the synthetic oligonucleotides, the method also allows for the immobilization of labeled (e.g., biotinylated) oligonucleotides, if desired. The amount of oligonucleotide that can be immobilized in a single well of an ELISA plate by this method is at least 500 (moles. The oligonucleotides thus immobilized onto the solid phase can hybridize to suitable templates and also participate in enzymatic reactions like template-directed extensions and ligations.

For high volume testing applications, it is desirable to use non-radioactive detection methods. Thus, the use of haptenated dideoxynucleotides is preferred; the use of biotinylated dideoxynucleotides is particularly preferred as such modification would render the incorporated base detectable by the standard avidin (or streptavidin) enzyme conjugates used in ELISA assays. The biotinylated ddNTPs are preferably prepared by reacting the four respective (3-aminopropyn-1-yl)nucleoside triphosphates with sulfosuccinimidyl 6-(biotinamido)hexanoate. Thus, (3-aminopropyn-1-yl) nucleoside 5′-triphosphates are prepared as described by Hobbs, F. W. (J. Org. Chem. 54:3420-3422 (1989)) and by Hobbs, F. W. et al. (U.S. Pat. No. 5,047,519). The (3-aminopropyn-1-yl)nucleoside 5′-triphosphate (50 mol) is dissolved in 1 ml of pH 7.6, 1 M aqueous triethylammonium bicarbonate (TEAB). Sulfosuccinimidyl 6-(biotinamido) hexanoate sodium salt (Pierce, 55.7 mg, 100 mol) is added and the solution is heated to 50° C. in a stoppered tube for 2 hr. The reaction mixture is diluted to 10 ml with water and applied to a DEAE-Sephadex A-25-120 column (1.6×19 cm). The column is eluted with a linear gradient of pH 7.6 aqueous TEAB (0.1 M to 1.0 M) and the eluent monitored at 270 nm. The late-eluting major peak is collected, stripped, and co-evaporated with ethanol. The crude product, containing biotinylated nucleoside triphosphate and, in some cases, contaminating starting material, is further purified by reverse phase column chromatography (Baker C-18 packing, 2×12 cm bed). The material is loaded in 0.1 M pH 7.6 TEAB and eluted with a step gradient of acetonitrile in 0.1 M pH 7.6 TEAB (0% to 36%, 2% increments, 8 ml/step). In all cases, the biotinylated product is more strongly retained and cleanly resolved from the starting material. Product-containing fractions are pooled, stripped, and co-evaporated with ethanol. The product is taken up in water and the yield calculated using the absorption coefficient for the starting nucleotide. The ¹H NMR and ³¹P NMR spectra are consistent with the expected structure and confirm the absence of phosphorus containing or nucleotide-derived impurities. The materials are observed to be >99% pure by HPLC (Waters Bondapak C-18, 4.6×250 mm, 1 ml/min, 1 to 35% CH₃CN/pH 7/0.01 M triethylammonium acetate).

### F. Solid Phase Analysis of Polymorphic Sites

### 1. Polymerase-Mediated Analysis

Although the identity of the nucleotide(s) of the polymorphic sites can be determined in a variety of ways, an especially preferred method exploits the oligonucleotide-based diagnostic assay of nucleic acid sequence variation disclosed by Goelet, P. et al. (PCT Application WO92/15712, herein incorporated by reference). In this assay, a purified oligonucleotide having a defined sequence (complementary to an immediate proximal or distal sequence of a polymorphism) is bound to a solid support, especially a microtiter dish. A sample, suspected to contain the target molecule, or an amplification product thereof, is placed in contact with the support, and any target molecules present are permitted to hybridize to the bound oligonucleotide.

In one preferred embodiment, an oligonucleotide having a sequence that is complementary to an immediately distal sequence of a polymorphism is prepared using the above-described methods (and preferably that of Nikiforov, T. (U.S. patent application Ser. No. 08/005,061). The terminus of the oligonucleotide is attached to the solid support, as described, for example by Goelet, P. et al. (PCT Application WO 92/15712), such that the 3′-end of the oligonucleotide can serve as a substrate for primer extension.

The immobilized primer is then incubated in the presence of a DNA molecule (preferably a genomic DNA molecule) having a single nucleotide polymorphism whose immediately 3′-distal sequence is complementary to that of the immobilized primer. Preferably, such incubation occurs in the complete absence of any dNTP (i.e. dATP, dCTP, dGTP, or dTTP), but only in the presence of one or more chain terminating nucleotide triphosphate derivatives (such as a dideoxy derivative), and under conditions sufficient to permit the incorporation of such a derivative on to the 3′-terminus of the primer. As will be appreciated, where the polymorphic site is such that only two or three alleles exist (such that only two or three species of dNTPs, respectively, could be incorporated into the primer extension product), the presence of unusable nucleotide triphosphate(s) in the reaction is immaterial. In consequence of the incubation, and the use of only chain terminating nucleotide derivatives, a single dideoxynucleotide is added to the 3′-terminus of the primer. The identity of that added nucleotide is determined by; and is complementary to, the nucleotide of the polymorphic site of the polymorphism.

In this embodiment, the nucleotide of the polymorphic site is thus determined by assaying which of the set of labeled nucleotides has been incorporated onto the 3′-terminus of the bound oligonucleotide by a primer-dependent polymerase. Most preferably, where multiple dideoxynucleotide derivatives are simultaneously employed, different labels will be used to permit the differential determination of the identity of the incorporated dideoxynucleotide derivative.

### 2. Polymerase/Ligase-Mediated Analysis

In an alternative embodiment, the identity of the nucleotide of the polymorphic site is determined using a polymerase/ligase-mediated process. As in the above embodiment, an oligonucleotide primer is employed, that is complementary to the immediately 3′-distal invariant sequence of the SNP. A second oligonucleotide, is tethered to the solid phase via its 3′-end. The sequence of this oligonucleotide is complementary to the 5′-proximal sequence of the polymorphism being analyzed, but is incapable of hybridizing to the oligonucleotide primer.

These oligonucleotides are incubated in the presence of DNA containing the single nucleotide polymorphism that is to be analyzed, and at least one 2′,5′-deoxynucleotide triphosphate. The incubation reaction further includes a DNA polymerase and a DNA ligase.

The tethered and soluble oligonucleotides are thus capable of hybridizing to the same strand of the single nucleotide polymorphism under analysis. The sequence considerations cause the two oligonucleotides to hybridize to the proximal and distal sequences of the SNP that flank the polymorphic site (X) of the polymorphism; the hybridized oligonucleotides are thus separated by a “gap” of a single nucleotide at the precise position of the polymorphic site.

The presence of a polymerase and a 2′,5′-deoxynucleotide triphosphate complementary to (X) permits ligation of the primer extended with the complementary 2′,5′-deoxynucleotide triphosphate to the immobilized oligo complementary to the distal sequence, a 2′,5′-deoxynucleotide triphosphate that is complementary to the nucleotide of the polymorphic site permits the creation of a ligatable substrate. The ligation reaction immobilizes the 2′,5′-deoxynucleotide and the previously soluble primer oligonucleotide to the solid support.

The identity of the polymorphic site that was opposite the “gap” can then be determined by any of several means. In a preferred embodiment, the 2′,5′-deoxynucleotide triphosphate of the reaction is labeled, and its detection thus reveals the identity of the complementary nucleotide of the polymorphic site. Several different 2′,5′-deoxynucleotide triphosphates may be present, each differentially labeled. Alternatively, separate reactions can be conducted, each with a different 2′,5′-deoxynucleotide triphosphate. In an alternative sub-embodiment, the 2′,5′-deoxynucleotide triphosphates are unlabeled, and the second, soluble oligonucleotide is labeled. Separate reactions are conducted, each using a different unlabeled 2′,5′-deoxynucleotide triphosphate. The reaction that contains the complementary nucleotide permits the ligatable substrate to form, and is detected by detecting the immobilization of the previously soluble oligonucleotide.

### G. Signal-Amplification

The sensitivity of nucleic acid hybridization detection assays may be increased by altering the manner in which detection is reported or signaled to the observer. Thus, for example, assay sensitivity can be increased through the use of detectably labeled reagents. A wide variety of such signal amplification methods have been designed for this purpose. Kourilsky et al. (U.S. Pat. No. 4,581,333) describe the use of enzyme labels to increase sensitivity in a detection assay. Fluorescent labels (Albarella et al., EP 144914), chemical labels (Sheldon III et al., U.S. Pat. No. 4,582,789; Albarella et al., U.S. Pat. No. 4,563,417), modified bases (Miyoshi et al., EP 119448), etc. have also been used in an effort to improve the efficiency with which hybridization can be observed.

It is preferable to employ fluorescent, and more preferably chromogenic (especially enzyme) labels, such that the identity of the incorporated nucleotide can be determined in an automated, or semi-automated manner using a spectrophotometer.

### IV. The Use of SNP Genotyping in Methods of Genetic Analysis

A. Using Single Nucleotide Polymorphisms in Genetic Analysis

The utility of the polymorphic sites located using the DNA of the present invention stems from the ability to use such sites to predict the statistical probability that two individuals will have the same alleles for any given polymorphisms.

Statistical analysis of SNPs can be used for any of a variety of purposes. Where a particular animal has been previously tested, such testing can be used as a “fingerprint” with which to determine if a certain animal is, or is not that particular animal.

Where a putative parent or both parents of an individual have been tested, the methods of the present invention may be used to determine the likelihood that a particular animal is or is not the progeny of such parent or parents. Thus, the detection and analysis of SNVs can be used to exclude paternity of a male for a particular individual (such as a stallion's paternity of a particular foal), or to assess the probability that a particular individual is the progeny of a selected female (such as a particular foal and a selected mare).

As indicated below, the present invention permits the construction of a genetic map of a target species. Thus, the particular array of polymorphisms identified by the methods of the present invention can be correlated with a particular trait, in order to predict the predisposition of a particular animal (or plant) to such genetic disease, condition, or trait. As used herein, the term “trait” is intended to encompass “genetic disease,” “condition,” or “characteristics.” The term, “genetic disease” denotes a pathological state caused by a mutation, regardless of whether that state can be detected or is asymptomatic. A “condition” denotes a predisposition to a characteristic (such as asthma, weak bones, blindness, ulcers, cancers, heart or cardiovascular illnesses, skeleto-muscular defects, etc.). A “characteristic” is an attribute that imparts economic value to a plant or animal. Examples of characteristics include longevity, speed, endurance, rate of aging, fertility, etc.

B. Identification and Parentage Verification

The most useful measurements for determining the power of an identification and paternity testing system are: (i) the “probability of identity” (p(ID)) and (ii) the “probability of exclusion” (p(exc)). The p(ID) calculates the likelihood that two random individuals will have the same genotype with respect to a given polymorphic marker. The p(exc) calculates the likelihood, with respect to a given polymorphic marker, that a random male will have a genotype incompatible with him being the father in an average paternity case in which the identity of the mother is not in question. Since single genetic loci, including loci with numerous alleles such as the major histocompatibility region, rarely provide tests with adequate statistical confidence for paternity testing, a desirable test will preferably measure multiple unlinked loci in parallel. Cumulative probabilities of identity or non-identity, and cumulative probabilities of paternity exclusion are determined for these multi-locus tests by multiplying the probabilities provided by each locus.

C. Gene Mapping and Genetic Trait Analysis Using SNPs

The polymorphisms detected in a set of individuals of the same species (such as humans, horses, etc.), or of closely related species, can be analyzed to determine whether the presence or absence of a particular polymorphism correlates with a particular trait.

To perform such polymorphic analysis, the presence or absence of a set of polymorphisms (i.e. a “polymorphic array”) is determined for a set of the individuals, some of which exhibit a particular trait, and some of which exhibit a mutually exclusive characteristic (for example, with respect to horses, brittle bones vs. non-brittle bones; maturity onset blindness vs. no blindness; predisposition to asthma, cardiovascular disease vs. no such predisposition). The alleles of each polymorphism of the set are then reviewed to determine whether the presence or absence of a particular allele is associated with the particular trait of interest.

Any such correlation defines a genetic map of the individual's species. Alleles that do not segregate randomly with respect to a trait can be used to predict the probability that a particular animal will express that characteristic. For example, if a particular polymorphic allele is present in only 20% of the members of a species that exhibit a cardiovascular condition, then a particular member of that species containing that allele would have a 20% probability of exhibiting such a cardiovascular condition. As indicated, the predictive power of the analysis is increased by the extent of linkage between a particular polymorphic allele and a particular characteristic. Similarly, the predictive power of the analysis can be increased by simultaneously analyzing the alleles of multiple polymorphic loci and a particular trait. In the above example, if a second polymorphic allele was found to also be present in 20% of members exhibiting the cardiovascular condition, however, all of the evaluated members that exhibited such a cardiovascular condition had a particular combination of alleles for these first and second polymorphisms, then a particular member containing both such alleles would have a very high probability of exhibiting the cardiovascular condition.

The detection of multiple polymorphic sites permits one to define the frequency with which such sites independently segregate in a population. If, for example, two polymorphic sites segregate randomly, then they are either on separate chromosomes, or are distant to one another on the same chromosome. Conversely, two polymorphic sites that are co-inherited at significant frequency are linked to one another on the same chromosome. An analysis of the frequency of segregation thus permits the establishment of a genetic map of markers.

The resolution of a genetic map is proportional to the number of markers that it contains. Since the methods of the present invention can be used to isolate a large number of polymorphic sites, they can be used to create a map having any desired degree of resolution.

The sequencing of the polymorphic sites greatly increases their utility in gene mapping. Such sequences can be used to design oligonucleotide primers and probes that can be employed to “walk” down the chromosome and thereby identify new marker sites (Bender, W. et al., J. Supra. Molec. Strut. 10(suppl.):32 (1979); Chinault, A. C. et al., Gene 5:111-126 (1979); Clarke, L. et al., Nature 287:504-509 (1980)).

The resolution of the map can be further increased by combining polymorphic analyses with data on the phenotype of other attributes of the plant or animal whose genome is being mapped. Thus, if a particular polymorphism segregates with brown hair color, then that polymorphism maps to a locus near the gene or genes that are responsible for hair color. Similarly, biochemical data can be used to increase the resolution of the genetic map. In this embodiment, a biochemical determination (such as a serotype, isoform, etc.) is studied in order to determine whether it co-segregates with any polymorphic site. Such maps can be used to identify new gene sequences, to identify the causal mutations of disease, for example.

Indeed, the identification of the SNPs of the present invention permits one to use complimentary oligonucleotides as primers in PCR or other reactions to isolate and sequence novel gene sequences located on either side of the SNP. The invention includes such novel gene sequences. The genomic sequences that can be clonally isolated through the use of such primers can be transcribed into RNA, and expressed as protein. The present invention also includes such protein, as well as antibodies and other binding molecules capable of binding to such protein.

The invention is illustrated below with respect to two of its embodiments—horses and humans. However, because the fundamental tenets of genetics apply irrespective of species, such illustration is equally applicable to any other species. Those of ordinary skill would therefore need only to directly employ the methods of the above invention to isolate SNPs in any other species, and to thereby conduct the genetic analysis of the present invention.

As indicated above, LOD scoring methodology has been developed to permit the use of RFLPs to both track the inheritance of genetic traits, and to construct a genetic map of a species (Lander, S. et al., Proc. Natl. Acad. Sci. (U.S.A.) 83:7353-7357 (1986); Lander, S. et al., Proc. Natl. Acad. Sci. (U.S.A.) 84:2363-2367 (1987); Donis-Keller, H. et al., Cell 51:319-337 (1987); Lander, S. et al., Genetics 121:185-199 (1989)). Such methods can be readily adapted to permit their use with the polymorphisms of the present invention. Indeed, such polymorphisms are superior to RFLPs and STRs in this regard. Due to the frequency of SNPs, it is possible to readily generate a dense genetic map. Moreover, as indicated above, the polymorphisms of the present invention are more stable than typical (VNTR-type) RFLP polymorphisms,

The polymorphisms of the present invention comprise direct genomic sequence information and can therefore be typed by a number of methods. In an RFLP or STR-dependent map, the analysis must be gel-based, and entail obtaining an electrophoretic profile of the DNA of the target animal. In contrast, an analysis of the polymorphisms (SNPs) of the present invention may be performed using spectrophotometric methods, and can readily be automated to facilitate the analysis of large numbers of target animals.

What is claimed is:

1. Substantially purified and isolated human adult caucasian male genomic DNA having the following STR marker profile:

| Amel | D3S1358 | VWA | FGA | D8S1179 | D21S11 | D18S51 |
|---|---|---|---|---|---|---|
| M | 15-17 | 16-17 | 21-25 | 12-14 | 29-32.2 | 17-18 |
| D5S818 | D13S317 | D7S820 | CSF1PO | TPOX | TH01 | D16S539 |
|---|---|---|---|---|---|---|
| 11-12 | 11-12 | 8-8 | 10-11 | 8-8 | 7-9.3 | 10-13 |

2. An isolated culture of cells comprising the human genomic DNA of claim 1.

3. A culture of cells according to claim 2, wherein said culture is homogeneous.

4. A culture of cells according to claim 2, wherein said culture is heterogeneous.

5. A non-human transgenic mammal containing the human genomic DNA of claim 1.
